(19) United States
(12) Patent Application Publication
(10) Pub. No.: US 2011/0178456 A1
(43) Pub. Date: Jul. 21, 2011

(54) ASSISTING TRANSDERMAL DRUG DELIVERY BY MEANS OF TISSUE FREEZING, VACUUM PRESSURE AND PHOTOHEATING PROCEDURES

(75) Inventors: Guillermo Aguilar-Mendoza, Corona, CA (US); Feng Sun, Riverside, CA (US); Bernard Choi, Irvine, CA (US)

(73) Assignee: THE REGENTS OF THE UNIVERSITY OF CALIFORNIA, Oakland, CA (US)

(21) Appl. No.: 12/753,785

(22) Filed: Apr. 2, 2010

Related U.S. Application Data
(60) Provisional application No. 61/166,668, filed on Apr. 3, 2009.

(51) Int. Cl. A61M 37/00; A61M 35/00

(52) U.S. Cl. 604/20; 604/290; 604/291

(57) ABSTRACT

This disclosure relates generally to methods for increasing the permeability of tissue. More particularly, it relates to increasing the permeability of a skin tissue or other epidermal tissue to promote delivery of active substances locally and systemically, and to control the delivery of active substances topically.

### CROSS REFERENCE TO RELATED APPLICATIONS

This application claims priority under 35 U.S.C. §119 from Provisional Application Ser. No. 61/166,668, filed Apr. 3, 2009, the disclosure of which is incorporated herein by reference.

### TECHNICAL FIELD

This disclosure relates generally to methods for increasing the permeability of tissue. More particularly, it relates to increasing the permeability of a skin tissue or other epidermal tissue to promote delivery of active substances locally and systemically, and to control the delivery of active substances topically.

### BACKGROUND

Poor permeation of many active substances into and through the skin often limits the utility of the topical route of administration and of topical formulations of active substances. Various methods exist in the art for increasing the permeability of the skin or for increasing the ability of an active substance to permeate the skin. Chemical enhancers can be used to reduce the barrier function of the skin or to alter the properties of the active substance so as to allow the active substance to better partition into the skin. These chemical modifiers can be quite irritating to the skin, and may not increase permeability adequately to allow therapeutic levels of many active substances to permeate the skin.

### SUMMARY

Use of tissue freezing either through refrigerants or cryogens can significantly assist transdermal drug delivery. The disclosure demonstrates effective delivery of large biomolecules by freezing the tissue prior to the application of pressure that deforms the tissue (e.g., positive or negative pressure) is useful for biomolecule delivery transdermally to a subject. Partially freezing of the stratum corneum and upper epidermis of skin and later exposing them to mechanical deformation (and in some embodiments photothermal/radiofrequency heating), create several pathways (cracks or microfissures) on the stratum corneum and even upper epidermis layers, that enhance the penetration of topical creams or topical agents.

The disclosure provides a method of trans-dermal delivery of a biomolecule comprising: freezing a location on the skin of a subject; contacting the location with a pressure to cause deformation of the skin; and contacting the location with an agent to be delivered. The contacting with the agent may be prior to, simultaneously with or after contacting with a freezing agent or a means for causing deformation of the skin. In one embodiment, the method further comprises contacting the location with heat prior to, simultaneously with, or immediately after contacting the location with the agent. In yet another embodiment, the pressure is negative pressure such as by a vacuum device. In one embodiment, the method promotes the generation of microcracks, fissures, channels or crevices (all used interchangeably herein) for permeating the skin's barrier and delivering an agent to a subject. In one embodiment, the micro-cracks/channels extend to a depth below the stratum corneum. In yet another embodiment, the pressure may be alternating positive and negative pressure. In yet another embodiment, the agent is an optical clearing agent.

The disclosure also provides a device for performing the method described above. In one embodiment, the device comprises a chamber the fittably seals over a subject's skin, wherein the chamber comprises a plurality of ports, wherein at least one port can be used to deliver a cryogenic agent for freezing the skin and another port can be used for delivery a pressure or biomolecule to the skin. In yet another embodiment, the device is computer controlled to adjust the pressure and timing of delivery of the cryogenic agent, drug or pressure. The device may further comprise a light source or laser for dermatological treatment.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 is a cartoon depicting the cross section of a subject's skin. (1) is the Stratum Corneum comprising kerotinocytes (3) and matrix material (2).

FIG. 2 depicts a process of the disclosure and micrographs of actual results obtained.

FIG. 3 are graphs depicting delivery of various sized biomolecules using the process of the disclosure compared to controls.

FIG. 4 shows results of 4 kDa and 20 kDa biomolecule delivery.

FIG. 5 further depicts the methods of the disclosure and results obtained.

### DETAILED DESCRIPTION

As used herein and in the appended claims, the singular forms “a,” “and,” and “the” include plural referents unless the context clearly dictates otherwise. Thus, for example, reference to “a factor” includes a plurality of such cells and reference to “the factors” includes reference to one or more primers and equivalents thereof known to those skilled in the art, and so forth.

Unless defined otherwise, all technical and scientific terms used herein have the same meaning as commonly understood to one of ordinary skill in the art to which this disclosure belongs. Although any methods and reagents similar or equivalent to those described herein can be used in the practice of the disclosed methods and compositions, the exemplary methods and materials are now described.

All publications mentioned herein are incorporated herein by reference in full for the purpose of describing and disclosing the methodologies, which are described in the publications, which might be used in connection with the description herein. The publications discussed above and throughout the text are provided solely for their disclosure prior to the filing date of the present application. Nothing herein is to be construed as an admission that the inventors are not entitled to antedate such disclosure by virtue of prior disclosure.

The disclosure demonstrates effective delivery of large biomolecules by freezing the tissue prior to the application of pressure that deforms the tissue (e.g., positive or negative pressure. Partially freezing of the stratum corneum and upper epidermis of skin and later exposing them to mechanical deformation (and in some embodiments photothermal/radiofrequency heating), create several pathways (cracks) on the stratum corneum and even upper epidermis layers, that enhance the penetration of topical creams.

“Active substance”, “active agent”, “agent”, “drug”, “biomolecule” and “substance” are used interchangeably and are intended to have their broadest interpretation as to any molecule or combination of molecules which is delivered to a living organism to produce a desired effect, such as for example a cosmetic or aesthetic effect. Any number of drugs may be used in the methods and systems of the disclosure. For example, the disclosure demonstrates that the range of drug surrogates in terms of their molecular weight (from 500 Daltons to 70,000 Daltons) and also in terms of its hydrophilicity can be used. The disclosure demonstrates that FITC (500 Daltons), and several combinations of FITC adhered to Dextran of various molecular weights from 4 kDa to 70 kDa have been effectively delivered. In addition, Dylight and Curcumin, which have different hydrophilicity, have been delivered.

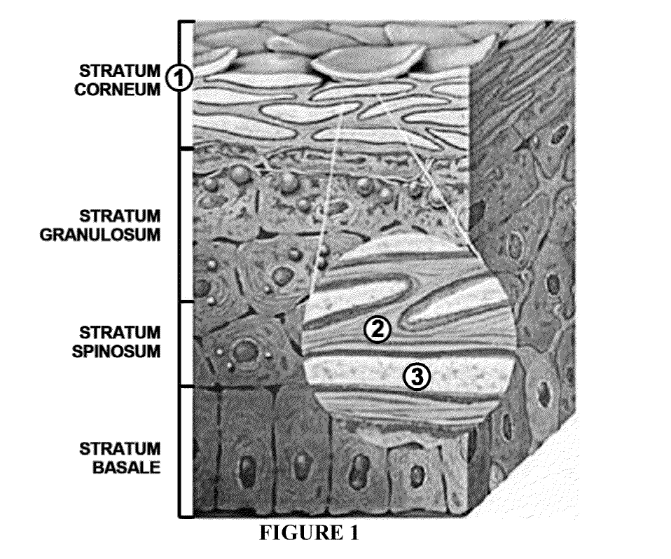

The disclosure provides a method of “cryo-cracking” of the epidermal layer of a tissue (e.g., a skin tissue) to promote permeability by inducing fissures, micro-cracks or channels in the stratum corneum. These fissures, microcracks or channels can penetrate through one or more layers of the epidermis (e.g., through the stratum corneum, see FIG. 1). Typically the cryo-cracking methods will not remove the layer of stratum corneum at a treated location. Cryo-cracking generally includes contacting a treatment location with a cryogenic agent followed by deformation of the location (e.g., stretching) to cause a disruption or bending of the treatment location. The deformation may be performed by pressure (e.g., positive pressure, negative pressure or a combination thereof).

A “cryogenic agent” refers to a chemical, coolant, gas or other agent that reduces the temperature of a tissue upon contact. Such cryogenic agents can be used to reduce the temperature range of a tissue from less than freezing (20 Degrees Celsius) to very low temperatures (−200 degrees Celsius). A wide range of cooling compounds (cryogenic agents) can be used, including, but not limited to gaseous, liquid or solid compounds, including refrigerants and cryogens. For example R134a (1,1,1,2 tetrafluor-oethane), dichlorodifluoromethane (CCl2 F2), chlorodifluoromethane (CHClF2), liquid nitrogen, cold water, ice, cold air and any compound capable of cooling skin to near or below freezing temperatures can be used. Thermoelectric coolers and other cooling devices can be used as well.

Exposure of an epidermal tissue (e.g., a skin tissue) to a cryogenic agent can range from minutes to millisecond exposures, depending on the type of coolant used. For example, R-134 has a temperature of −26° C. and in specific methods of the disclosure was used in 50-500 millisecond bursts to cool skin to −10 to −15° C.

The disclosure demonstrates the effective “cracking” of the stratum corneum produced by the combined use of cold fluid on the skin followed by the stretching induced by a suction cup (˜50 kPa pressure for about 1 sec in 3 cycles with 1 sec. delay) can open the stratum corneum barrier for drug delivery.

The stretching can be caused by positive pressure or negative pressure. The pressure can be a mechanical pressure on the skin of a subject or may be the result of an air blast or other physical pressure. The stretching typically will cause a deformation of the skin by about 100 microns to several millimeters and can be adjusted using various devices in the art.

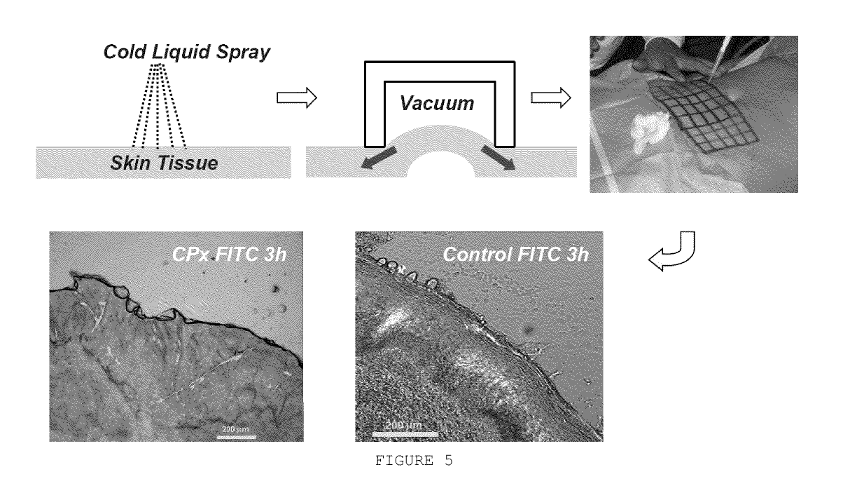

The disclosure also demonstrates that even 3 hrs after topical application using cryo-cracking procedure, the penetration of FITC into the stratum corneum and epidermis down the microcracks is much more significant compared to the penetration after simple rubbing at the same time (see, e.g., FIG. 5).

“Dermis” refers to the layer of the skin below the epidermis that typically contains blood capillaries, blood vessels, lymph vessels, hair follicles, and various glands. The dermis is divided into the upper or papillary layer and the lower or reticular layer

“Effective amount” is a dosage sufficient to produce a desired result. The desired result may comprise a subjective or objective improvement in the patient that receives the dosage.

“Epidermis” refers to the upper or outer nonvascular layers of the skin, including the stratum corneum, stratum lucidum, stratum granulosum, stratum spinosum, and stratum basale layers.

A “fissure”, “micro-crack”, or “channel” refers to a small opening in a surface. For the purposes of disclosure, a fissure, micro-crack or channel created in the stratum corneum by freezing and positive, negative or combination pressure will be understood as meaning a small opening that does not remove the layers of the stratum corneum. Typically the micro-crack is about 25 microns to 500 microns or more (e.g., about 0.1, 0.2, 0.3, 0.4, 0.5, 0.6, 0.7. 0.8, 0.9 to about 1 mm) in length and extends through the stratum corneum but not the stratum granulosum. However, in certain instances it may be desirable to increase the depth of the crack through further layers of the skin. In one embodiment, the microcrack is from about 50 microns to 200 microns and extends across the stratum corneum.

“Skin” refers generally to the body's outer covering, and includes the epidermis, dermis and subcutis.

“Stratum corneum” refers to the horny outer layer of the epidermis, consisting of several layers of flat, keratinized non-nucleated dead or peeling cells, with naturally occurring pores interspersed in the tissue.

“Subcutis” and “subcutaneous tissue” are used interchangeably and refer to the layer of tissue directly under the dermis. The subcutis is composed mainly of adipose tissue, and separates the dermis from the underlying muscle.

“Substantially intact stratum corneum” refers to a stratum corneum that comprises fissures, micro-cracks or channels produced by freezing, radiowaves, or a combination of freezing and pressure or radiowaves, wherein the stratum corneum remains physically present following treatment.

“Therapy” and “treatment” are used interchangeably and include, but are not limited to, changes in a subject's status. The changes can be either subjective or objective and can relate to features such as symptoms or signs of the disease or condition being treated or the presence of a drug or biomolecule delivered to the subject (e.g., transdermally). For example, if the subject notes improvements in a dermatological condition, improvements in skin appearance, reduced discomfort or decreased pain, then successful treatment has occurred. Similarly, if the clinician notes objective changes, such as by histological analysis of a biopsy sample, then treatment has also been successful. Alternatively, the clinician may note a decrease in the size of lesions or other abnormalities upon examination of the subject. This would also represent an improvement or a successful treatment. Preventing the deterioration of a patient's status is also included by the term. Therapeutic benefit includes any of a number of subjective or objective factors indicating a response of the condition being treated, or an improvement in skin appearance, as discussed herein.

A “therapeutically effective amount” refers to the amount of an active substance sufficient to induce a desired biological result. That result may be alleviation of the signs, symptoms, and/or causes of a disease, or any other desired alteration of a biological system. The term “therapeutically effective amount” is used herein to denote any amount of the formulation which causes a substantial improvement in a disease condition when applied to the affected areas once or repeatedly over a period of time in a clinically reasonable manner. The amount will vary with the condition being treated, the stage of advancement of the condition, and the type and concentration of formulation applied. Appropriate amounts in any given instance will be readily apparent to those skilled in the art or capable of determination by routine experimentation.

A “treated location” refers to the portion of tissue which is contacted during treatment with a cooling agent, freezing agent, radiowaves and/or pressure.

The methods can be conducted using positive pressure, negative pressure or a combination of alternating pressures to increase the size of fissures, micro-cracks or channels created in the stratum corneum and which can also increase the rate and amount of active substance that permeates the skin. Devices for providing such pressures are known in the art (e.g., vacuum hoses, and devices, pressure waves etc.). Specifically, a means for providing increased pressure in the range of about 1 to 30 pounds per square inch above atmospheric pressure can be placed against the skin before, during or after contact with a cryogenic agent to increase the permeability of the skin and/or the uptake of an active substance by the skin.

In another embodiment, the cryo-cracking methods described herein can be used generally to increase the permeability of the skin so as to allow the permeation of a wide range of active substances into and through the skin. The cryo-cracking methods and devices of the disclosure are useful for delivering one, or more than one, active substance to the layers of skin below the stratum corneum, for treatment of skin or tissue conditions. These treatments can also be used to deliver active substances to the layers of skin below the stratum corneum so as to deliver active substances into the general circulation, for treatment of local or systemic conditions. The active substance can be a substance or factor that is beneficial to the subject and/or treats a condition present in the subject. The active substance can be in the form of a cosmetic composition and/or a pharmaceutical composition. The active substance can be delivered to provide a prophylactic treatment, a cosmetic treatment and/or a therapeutic treatment. The active substance can be applied once, repeatedly or continuously to the tissue before, during and/or after a cryo-cracking treatment.

The cryo-cracking methods described herein can be used for controlled delivery of active substances. Specifically, by creating pores, fissures, micro-cracks, or channels in the skin using the methods described herein and applying active substances to the treated skin, the rates at which active substances permeate through the treated skin and into deeper tissue and the systemic circulation can be controlled. For example, formulations containing encapsulated active substances (e.g., active substances encapsulated in liposomes, niosomes, ethosomes, transfersomes, microspheres, etc.) can be applied topically to fractionally treated skin, and the permeation of the encapsulated active substance can be controlled.

In another embodiment, the treatments described herein can be used to deliver active substances into the skin that would not normally permeate the skin to any measurable degree without first generating pores, fissures, micro-cracks or channels in the skin. In one example, the techniques described herein can be used to deliver a photodynamic substance into the skin, as well as to control the depth to which the photodynamic substance is applied within the skin. For example, a photodynamic substance can be an optical clearing agent or an agent that absorbs electromagnetic light at a particular wavelength.

Optical clearing agents are compounds used to increase the optical transmission properties of skin, which allows for more effective laser treatment of skin conditions. In dermatology, optical therapeutic and diagnostic methods are applied to treat or study diverse problems such as vascular birthmarks, psoriasis, and cancer, laser-assisted hair and tattoo removal, the treatment of scars, wrinkles, melasma, striae distensae and pigmentation in conjunction with fractional resurfacing. The complex morphological nature of human skin results in a highly scattering medium at visible and near-infrared wavelengths. Scattering diminishes the depth and clarity of images in optical diagnostic imaging and attenuates the effective light dose that reaches targeted structures in laser therapeutics. Optical diagnostic techniques and therapeutics can be improved if optical clearing agents are delivered thereby enhancing light penetration into human skin. While optical clearing agents have been identified that decrease tissue optical scattering and hence increase light penetrance, there is a need in the art for the safe and effective delivery of these agents into human skin.

Compositions that can be used as optical clearing agents include mixtures of polypropylene glycol and polyethylene glycol. Other compounds can be used, including sugar alcohols, organic solvents, organic acids, 50% TMP, TMP, 1-3-butanediol, Ethylene glycol, MPDiol glycol, DMSO, and dimethyl sulfoxide.

In one embodiment, following cryo-cracking, the photodynamic substance can be applied to the skin, allowed to penetrate into the pores/fissures created by the cryo-cracking, the unpermeated portion of the substance can be removed from the surface of the skin, and the permeated portion of the substance activated by an appropriate light source. Other photodynamic substance can be selected from the group consisting of a psoralen, methoxsalen, trioxsalen, 8-methoxy psoralen, porfimer sodium, aminolevulinic acid, and combinations thereof.

In another embodiment, the cryo-cracking methods and devices of the disclosure can be used in combination with other treatment for skin, including heating using lasers, electromagnetic radiation or radiowaves. In addition to producing increased skin permeability, an active substance can be applied in conjunction with a laser treatment to increase the permeation of the active substance, increase the benefits of the laser treatment, or increase the rate of recovery from the treatment.

The methods of the disclosure can be used to treat a variety of dermatological diseases and/or conditions. The dermatological disease and/or condition can include a pigmentary disorder, post-inflammatory hyperpigmentation, melasma, striae, scar tissue, and combinations thereof. When a cryo-cracking treatment as described herein is used in this manner, an active substance can be applied in conjunction with the treatment. The active substance can be selected from the group consisting of a vitamin, a mineral, an anti-oxidant, an agent that promotes skin recovery, and combinations thereof. The active substance can be selected from the group consisting of a drug for treatment of a pigmentary disorder, an agent for inducing collagen remodeling, a retinoid, a neurotoxin, an antibiotic and combinations thereof.

The dermatological disease and/or condition can include acne, rosacea, alopecia, neoplasia of the skin, and combinations thereof. When the cryo-cracking treatments disclosed herein can be used to treat acne, rosacea and combinations thereof, an active substance can be applied in conjunction with the treatment. The active substance can be selected from the group consisting of a drug for treatment of acne, a drug for treatment of rosacea, a vitamin, a mineral, an anti-oxidant, an agent that promotes skin recovery, an antibiotic and combinations thereof.

The treatments disclosed herein can be used to treat alopecia. When the treatments are used in this manner, an active substance can be applied in conjunction with the treatment. The active substance can be selected from the group consisting of a drug for treatment of alopecia, a vitamin, a mineral, an anti-oxidant, an agent that promotes skin recovery, an antibiotic and combinations thereof.

In another embodiment, the cryo-cracking methods and devices described herein can be used to treat a disease and/or condition selected from the group consisting of hypervascular lesions, port wine stains, capillary hemangiomas, cherry angiomas, venous lakes, poikiloderma of civate, angiokeratomas, spider angiomas, facial telangiectasias, telangiectatic leg veins, pigmented lesions, lentigines, ephelides, nevus of Ito, nevus of Ota, Hori's macules, keratoses pilaris, acne scars, epidermal nevus, Bowen's disease, actinic keratoses, actinic cheilitis, oral florid papillomatosis, seborrheic keratoses, syringomas, trichoepitheliomas, trichilemmomas, xanthelasma, apocrine hidrocystoma, verruca, adenoma sebacum, angiokeratomas, angiolymphoid hyperplasia, pearly penile papules, venous lakes, and combinations thereof. When the treatments are used in this manner, an active substance can be applied in conjunction with the treatment. The active substance can be selected from the group consisting of a drug, a vitamin, a mineral, an anti-oxidant, an agent that promotes skin recovery, an antibiotic and combinations thereof.

The cryo-cracking methods and devices disclosed herein can be used to treat a disease and/or condition selected from the group consisting of atopic dermatitis, psoriasis, a bacterial infection, a viral infection, a fungal infection, an infestation, a neoplasm of the skin, and combinations thereof. When the treatments are used in this manner, an active substance can be applied in conjunction with the treatment. The active substance can be selected from the group consisting of a drug for treatment of atopic dermatitis, a drug for treatment of psoriasis, an antibiotic, a drug for treatment of viral infections, a drug for treatment of fungal infections, a drug for treatment of infestations, a drug for treatment of neoplasms of the skin, a photodynamic substance, a vitamin, a mineral, an anti-oxidant, an agent that promotes skin recovery and combinations thereof.

When the treatments disclosed herein are used to deliver an active substance, the active substance can be in the form of an active substance in a carrier. The active substance can be in the form of a cosmetically effective amount of an active substance in a cosmetically acceptable carrier. The active substance can be a pharmaceutically effective amount of an active substance in a pharmaceutically acceptable carrier. The active substance can be a liquid or a semi-solid composition. The active substance can be a lotion, cream, gel or ointment. The active substance can be in the form of a paste, plaster or mask. The active substance can be in the form of a hydrogel or urethane foam infused with the active substance. The active substance can be in the form of a hydrogel or urethane mask.

When the treatments disclosed herein are used to deliver an active substance, the active substance can be a protein or peptide. When the active substance is a protein or peptide, the protein or peptide can be naturally occurring, recombinant, or synthetic. The protein or peptide can be composed of all of the amino acids present in the naturally occurring form of the protein or peptide or can be composed of an active subset of the amino acids present in the naturally occurring protein or peptide.

The active substance can be delivered by the treatment methods described herein can be a local anaesthetic. The local anaesthetic can be selected from the group consisting of benzocaine, bupivicaine, chloroprocaine, cocaine, etidocaine, lidocaine, mepivacaine, pramoxine, prilocalne, procaine, proparacaine, ropivicaine, tetracaine, and combinations thereof.

The active substance that can be delivered using the treatment methods described herein can be a drug for treatment of acne. The drug for treatment of acne can be selected from the group consisting of azelaic acid, benzoyl peroxide, clindamycin, erythromycin, tetracycline, trimethoprim, minicycline, doxycycline, metronidazole, sulfacetamine, sulfur, salicylic acid, a retinoid, spironolactone, cyproterone acetate, a glucocorticoid, an estrogen, a progestin, prednisone, dexamethasone, and combinations thereof.

The active substance that can be delivered can be a drug for treatment of rosacea. The drug for treatment of rosacea can be selected from the group consisting of a tetracycline antibiotic, tetracycline, doxycycline, minocycline, an antibiotic, metronidazole, a beta blocker, propanolol, an antihistamine, cetirizine, loratadine, and combinations thereof.

The active substance that can be delivered can be a drug to treat alopecia. The drug to treat alopecia can be selected from the group consisting of a calcium channel blocker, minoxidil, a 5-alpha reductase inhibitor, finasteride, dutasteride, a retinoid, and combinations thereof.

The drug for treatment of neoplasms of the skin can be selected from the group consisting of a retinoid, 5-fluorouracil, imiquimod, denileukin diftitox, mechlorethamine hydrochloride, carmustine, glucocorticosteroids, porfimer sodium, alpha-aminolevulinic acid, and combinations thereof.

The active substance that can be delivered can be a photodynamic substance. The photodynamic substance can be selected from the group consisting of a psoralen, methoxsalen, trioxsalen, 8-methoxy psoralen, porfimer sodium, aminolevulinic acid, and combinations thereof. Optical Clearing Agents are compounds used to increase the optical transmission properties of skin, which allows for more effective laser treatment of skin conditions. In dermatology, optical therapeutic and diagnostic methods are applied to treat or study diverse problems such as vascular birthmarks, psoriasis, and cancer, laser-assisted hair and tattoo removal, the treatment of scars, wrinkles, melasma, striae distensae and pigmentation in conjunction with fractional resurfacing. The complex morphological nature of human skin results in a highly scattering medium at visible and near-infrared wavelengths. Scattering diminishes the depth and clarity of images in optical diagnostic imaging and attenuates the effective light dose that reaches targeted structures in laser therapeutics. Optical diagnostic techniques and therapeutics can be improved if optical clearing agents are delivered thereby enhancing light penetration into human skin. While optical clearing agents have been identified that decrease tissue optical scattering and hence increase light penetrance, there is a need in the art for the safe and effective delivery of these agents into human skin

Compositions that can be used as optical clearing agents include mixtures of polypropylene glycol and polyethylene glycol. Other compounds can be used, including sugar alcohols, organic solvents, organic acids, 50% TMP, TMP, 1-3-butanediol, Ethylene glycol, MPDiol glycol, DMSO, and dimethyl sulfoxide.

The active substance that can be an antibiotic. The antibiotic can be selected from the group consisting of tetracycline, doxycycline, minocycline, erythromycin, trimethoprim, sulfamethoxazole, clindamycin, mupirocin, silver sulfadiazine, and combinations thereof.

The active substance that can be a retinoid. The retinoid can be selected from the group consisting of vitamin A, retinol, retinoic acid, tretinoin, isotreninoin, alitretionoin, etreinate, acitretin, an arotinoid, tazarotene, bexarotene, adapalene, Ro 13-7410, Ro15-1570, and combinations thereof.

The active substance that can be a neurotoxin. The neurotoxin can be selected from the group consisting of a neurotoxic compound produced by a form of Clostridia, a neurotoxic compound produced by Clostridium botulinum, a form of botulinum toxin, botulinum toxin type A, botulinum toxin type B, botulinum toxin type C, botulinum toxin type D, botulinum toxin type E, botulinum toxin type F, botulinum toxin type G, a botulinum neurotoxin peptide, a botulinum neurotoxin A (BoNT/A) peptide, a botulinum toxin in combination with a polysaccharide, a botulinum toxin in combination with a carrier comprising a polymeric backbone having attached positively charged branching groups, a botulinum toxin in combination with human serum albumin, a botulinum toxin in combination with a neuron growth inhibitor, a botulinum toxin in combination with a non-oxidizing amino acid derivative and zinc, a botulinum toxin in combination with a recombinant gelatin fragment, a stabilized botulinum toxin composition, and combinations thereof.

The active substance that can be a vitamin. The vitamin can be selected from the group consisting of a provitamin, a vitamin cofactor, a vitamin derivative, a form of vitamin A, a carotenoid, a retinoid, a form of B complex vitamin, thiamin, vitamin B1, riboflavin, nicotinic acid, vitamin B6, pyridoxine, pyridoxal, pyridoxamine, pantothenic acid, biotin, vitamin B12, a form of vitamin C, ascorbic acid, a form of vitamin D, a form of vitamin E, a tocopherol, a form of vitamin K, phylloquinone, a menanquinones, a form of carnitine, choline, folic acid, inositol, and combinations thereof. The vitamin can be selected from the group consisting of a form of vitamin C, a form of vitamin A, a form of vitamin E, and combinations thereof. The active substance that can be delivered by the cryo-cracking treatments described herein can be a mineral. The mineral can be selected from the group consisting of a trace mineral, calcium, copper, fluoride, iodine, iron, magnesium, phosphorus, selenium, zinc, and combinations thereof.

The active substance that can be an anti-oxidant. The anti-oxidant can be selected from the group consisting of a vitamin, a mineral, a hormone, a carotenoid terpenoid, a non-carotenoid terpinoid, a flavonic polyphenolic, a phenolic acid, an ester of a phenolic acid, a non-flavinoid phenolic, citric acid, a lignan, a phytoestrogen, oxalic acid, phytic acid, bilirubin, uric acid, a form of lipoic acid, silymarin, a form of acetylcystine, an emblicanin antioxidant, a free-radical scavenger, a peroxiredoxin, a form of catalase, a form of superoxide dismutase (SOD), a form of glutathione, a form of thioredoxin, a form of coenzyme Q, a bioflavinoid, a green tea extract, epigallo catechin gallate (EGCG), and combinations thereof.

The active substance that can be an agent that promotes skin recovery. The agent that promotes skin recovery can be selected from the group consisting of an interleukin, a chemokine, a leukotriene, a cytokine, myeloperoxidase, an antibiotic, a growth factor, a heat shock protein, a matrix metalloproteinase, a hormone, an estrogen, tea tree oil, and combinations thereof.

The active substance that can be an agent for treatment of the effects of ageing of the skin. The agent for treatment of the effects of aging of the skin can be an agent for treatment of the effects of photoaging and/or chronological aging. The agent for treatment of the effects of ageing of the skin can be selected from the group consisting of a vitamin, a mineral, an antioxidant, an agent to promote recovery, a growth factor, a cytokine, a heat shock protein, an agent to induce collagen remodeling, paeoniflorin, a form of an alpha hydroxyl acid, a form of a beta hydroxyl acid, a form of kinetin, a retinoid, a form of emu oil, a form of ubiquinone, and combinations thereof. The active substance that can be delivered by the cryo-cracking treatment methods described herein can be an agent to reduce the appearance of wrinkles in the skin. The agent to reduce the appearance of wrinkles in the skin can be selected from the group consisting of a vitamin, a mineral, an antioxidant, an agent to promote recovery, a growth factor, a cytokine, a heat shock protein, an agent to induce collagen remodeling, a humectant, a neurotoxin, a muscle relaxant, a form of an alpha hydroxyl acid, a form of a beta hydroxyl acid, an anti-oxidant, and combinations thereof.

The active substance that can be a growth factor. The growth factor can be naturally occurring, recombinant, or synthetic. The growth factor can be composed of all of the amino acids present in the naturally occurring form of the growth factor or can be composed of an active subset of the amino acids present in the naturally occurring growth factor. The growth factor can be selected from the group consisting of a colony stimulating factor (CSF), granulocyte-colony stimulating factor, (G-CSF), epidermal growth factor (EGF), erythropoietin (Epo), a fibroblast growth factor (FGF), FGF1, basic fibroblast growth factor, FGF2, FGF3, FGF4, a growth differentiation factor (GDF), myostatin, GDF8, GDF9, hepatocyte growth factor (HGF), an insulin-like growth factor (IGF), IGF-T, IGF-II, an interferon, INF-alpha, INF-beta, INF-gamma, leptin, nerve growth factor (NGF), a neurotropin, oncostatin (OSM), platelet-derived growth factor (PDGF), platelet growth factor (PGF), pleiotropin, thrombopoietin (TPO), a transforming growth factor (TGF), TGF-alpha, TGF-beta, a tumor necrosis factor (TNF), TNF-alpha, TNF-beta, vascular endothelial growth factor (VEGF), and combinations thereof. The growth factor can be selected from the group consisting of TGF-.beta., VEGF, EGF, leptin, a TGF, NGF, a neurotrophin, and combinations thereof.

The active substance that can be an agent for inducing collagen remodeling. The agent for inducing collagen remodeling can be selected from the group consisting of an endopeptidase, a zinc-dependent endopeptidase, a matrix metalloproteinase (MMP), a collagenase, a stromelysin, a matrilysin, a gelatinase, a contertase-activatable MMP, a membrane bound MMP, MMP-1, MMP-2, MMP-3, MMP-4, MMP-5, MMP-6, MMP-7, MMP-8, MMP-9, MMP-10, MMP-11, MMP-12, MMP-13, MMP-14, MMP-15, MMP-16, MMP-17, MMP-18, MMP-19, MMP-20, MMP-21, MMP-22, MMP-23, MMP-23A, MMP-23B, MMP-24, MMP-25, MMP-26, MMP-27, MMP-28, and combinations thereof.

The active substance that can be a cytokine. The cytokine can be selected from the group consisting of an autocrine cytokine, an endocrine cytokine, a paracrine cytokine, and combinations thereof. The active substances that can be delivered by the cryo-cracking treatments described herein can be an interleukin (IL). The interleukin can be selected from the group consisting of IL-1, IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20, IL-21, IL-22, and combinations thereof. The interleukin can be selected from the group consisting of IL-1, IL-2, and combinations thereof. The active substance that can be delivered by the cryo-cracking treatments described herein can be a heat shock protein (Hsp). Heat shock proteins, also known as stress proteins, are a group of proteins expressed when cells are exposed to elevated temperatures. The heat shock protein can be selected from the group consisting of a HspA group protein, Hsp70, Hsp71, Hsp72, Hsp78, a HspB group protein, HspB1, Hsp40, a HspC group protein Hsp90, glucose-regulated protein 94, Hsp10, Hsp20, Hsp25, Hsp27, Hsp42, Hsp60, Hsp90, Hsp100, Hsp104, Hsp110, binding immunoglobulin protein, a small heat shock protein, and combinations thereof.

The active substance that can be a drug for the treatment of a dermatological disease or condition. The drug for treatment of a dermatological condition can be selected from the group consisting of a drug for treatment of atopic dermatitis, a drug for treatment of psoriasis, a drug for photodynamic therapy, a drug for treatment of acne, an antibiotic, a drug for treatment of viral infections, a drug for treatment of fungal infections, a drug for treatment of infestations, a drug for treatment of neoplasms of the skin, a drug for treatment of alopecia, a drug for treatment of pigmentary disorders, and combinations thereof. The drug for treatment of pruritis can be selected from the group consisting of an antihistamine, menthol, camphor, phenol, pramoxine, doxepin, capsaicin, tar, a steroid, and combinations thereof. The drug for treatment of atopic dermatitis can be selected from the group consisting of a glucocorticosteroid, an antihistamine, a leukotriene receptor antagonist, an immunosuppressive agent, and combinations thereof. The drug for treatment of psoriasis can be selected from the group consisting of calcipotriene, anthralin, tazarotene, cytotoxic agents, acitretin, cyclosporine, mycophenolate mofetil, and combinations thereof. The drug for photodynamic therapy can be selected from the group consisting of a psoralen, methoxsalen, trioxsalen, 8-methoxy psoralen, porfimer sodium, aminolevulinic acid and combinations thereof. The drug for treatment of viral infections can be selected from the group consisting of acyclovir, famciclovir, valacyclovir, penciclovir, podophyllin, podofilox, imiquimod and combinations thereof. The drug for treatment of a fungal infection can be selected from the group consisting of an azole, fluconazole, ketaconazole, micronazole, itraconazole, econazole, econazole nitrate, an allylamine, naftifine, terbinafine, griseofulvin, ciclopirox and combinations thereof. The drug for treatment of infestations can be selected from the group consisting of gamma benzene hexachloride, lindane, premetrin, ivermectin, crotamition, sulfur, and combinations thereof. The drug for treatment of a pigmentary disorder can be selected from the group consisting of a quinolone, hydroquinolone, a corticosteroid, fluocinolone acetonide, a retinoid, a licorice extract, a bleaching agent, koijic acid, and combinations thereof.

The active substance that can be a drug for treatment of neoplastic diseases, including neoplastic diseases of the skin and other tissues. The drug for treatment of neoplastic diseases can be selected from the group consisting of an alkylating agent, an antimetabolite, a natural product, a hormone, a hormone antagonist, and combinations thereof. The drug for treatment of neoplastic diseases can be selected from the group consisting of a nitrogen mustard, an ethylenimine, a methylenaimine, an alkyl sulfonate, a nitrourea, a triazene, a folic acid analog, a pyrimidine analog, a purine analog, an inhibitor related to a purine analog, a vinca alkaloid, a taxane, an epipodophyllotoxin, a camptothecin, an antibiotic, an enzyme, a biological response modifier, a platinum coordination complex, an anthracenedione, a substituted urea, a methylhydralazine derivative, an adrenocortical suppressant, a tyrosine kinase inhibitor, an adrenocorticosteroid, a progestin, an estrogen, an anti-estrogen, an androgen, an anti-androgen, a gonadotropin-releasing hormone analog, and combinations thereof. The active substance that can be delivered by the cryo-cracking treatments described herein can be a drug for immunomodulation therapy. The drug for immunomodulation therapy can be selected from the group consisting of an immunomodulator, an immunosuppressive agent, a tolerogen, an immunostimulant, and combinations thereof. The active substance that can be delivered by the cryo-cracking treatments described herein can be a drug acting on the blood or on the blood-forming organs. The drug acting on the blood or on the blood-forming organs can be a hematopoietic agent, a growth factor, a mineral, a vitamin, an anticoagulant, a thrombolytic drug, an antiplatelet drug, and combinations thereof.

The active substance that can be a hormone, hormone agonist, or hormone antagonist. The hormone, hormone agonist or hormone antagonist can be selected from the group consisting of a pituitary hormone; a hypothalamic releasing factor; a form of thyroid; an antithyroid drug; an estrogen; a progestin; an androgen; a form of adrenalin; an adrenocorticotropic hormone; a adrenocortical steroid; a synthetic steroid analog; an inhibitor of the synthesis of an adrenocortical hormone; an inhibitor of the action of an adrenocortical hormone; insulin; an oral hypoglycemic agent; an agent affecting calcification; an agent affecting bone turnover, calcium, phosphorus, or vitamin D; calcitonin; parathyroid hormone; an analog of parathyroid hormone; an agonist of parathyroid hormone; an antagonist of parathyroid hormone; and combinations thereof. The active substance that can be delivered by the cryo-cracking treatment methods described herein can be a glucocorticoid. The glucocorticoid can be selected from the group consisting of betamethasone, betamethasone diproprionate, betamethasone valerate, clobetasol propionate, difluorasone diacetate, halobetasol propionate, actinomine, desoximetasone, fluocinonide, fluocinolone acetonide, flurandrenolide, hydrocortisone, hydrocortisone butyrate, hydrocortisone valerate, halcinonide, triamcinolone acetonide, amcinonide, mometasone furoate, aclometasone dipropionate, desonide, dexamethasone, dexamethasone sodium phosphate, and combinations thereof.

The active substance that can be an antihistamine. The antihistamine can be selected from the group consisting of doxepin hydrochloride, caribinoxamine maleate, clemastine fumarate, diphenhydramine hydrochloride, dimenhydrinate, pyrilaimine maleate, tripelennamine hydrochloride, tripelennamine citrate, chlorpheniramine maleate, brompheniramine maleate, hydroxyzine hycrochloride, hydroxyzine pamoate, cyclizine hydrochloride, cyclizine lactate, meclizine hydrochloride, promethazine hydrochloride, cyproheptadine hydrochloride, phenindamine tartrate, acrivastine, cetirizine hycrochloride, azelastine hycrocholride, lovocasastine hydrochloride, loratidine, fexofenadine, and combinations thereof.

The active substance that can be an anti-inflammatory drug. The anti-inflammatory drug can be selected from the group consisting of histamine, a histamine antagonist, bradykinin, a bradykinin antagonist, a lipid-derived autacoid, an eicosanoid, a platelet-activating factor, an analgesic-antipyretic agent, a cyclooxygenase-2 (COX-2) inhibitor, a drug for treatment of gout, a drugs for treatment of asthma, and combinations thereof. The anti-inflammatory agent can be a non-specific COX-2 inhibitor. The non-specific COX-inhibitor can be a salicylic acid derivative, aspirin, sodium salyclate, choline magnesium trisalicylate, salsalate, diflunisal, sulfasalazine, olsalazine, a para-aminophenol derivative, acetaminophen, an indole, an indene acetic acid, indomethacin, sulindac, a heteroaryl acetic acid, tolmetrin, diclofenac, ketorolac, a arylpropionic acid, ibuprofen, naproxen, flurbiprofen, ketoprofen, fenoprofen, oxaproxin, an anthranilic acid, mefenamic acid, meclofenamic acid, an enolic acid, an oxicam, proxicam, meloxicam, an alkonone, nabumetone, and combinations thereof. The anti-inflammatory agent can be a selective COX-2 inhibitor selected from the group consisting of a diaryl-substituted furanone, a diaryl-substituted pyrazole, an indole acetic acid, a sulfonanilide, and combinations thereof. The COX-2 inhibitor can be selected from the group consisting of celecoxib; rofecoxib; meloxicam; piroxicam; valdecoxib, parecoxib, etoricoxib, CS-502, JTE-522; L-745,337; FR122047; NS398; from non-selective non-steroidal anti-inflammatory agents that would include aspirin, ibuprofen, indomethacin CAY10404, diclofenac, ketoprofen, naproxen, ketorolac, phenylbutazone, tolfenamic acid, sulindac, and others, or from steroids or corticosteroids.

The active substance that can be a vasoconstrictor. The vasoconstrictor can be selected from the group consisting of an antihistamine, a form of adrenaline, a form of asymmetric dimethylarinine, a form of adenosine triphosphate (ATP), a catecholamine, cocaine, a decongestant, a form of diphenhydramine, a form of endothelin, a form of phenylephrine, a form of epinephrine, a form of pseudoephedrine, a form of neuropeptide Y, a form of norepinephrine, a form of tetrandrozoline, a form of thromboxane, and combinations thereof.

The active substance that can be a drug that acts at synaptic and neuroeffector junctional sites. The drug that acts at a synaptic and neuroeffector junctional site can be selected from a neurotransmitter, a muscarinic receptor agonist, a muscarinic antagonist, an anticholinerase agent, an agent acting on the neuromuscular junction and autonomic ganglia, a catecholamine, a sympathomimetic drug, an adrenergic receptor agonist, an adrenergic receptor antagonist, a serotonin receptor agonist, a serotonin receptor antagonist, and combinations thereof. The active substance that can be delivered by the cryo-cracking treatments described herein can be a drug that acts on the central nervous system. These drug that acts on the central nervous system can be a general anaesthetic, a local anaesthetic, a therapeutic gas, a hypnotic, a sedatives, a drug for treatment of depression, a drug for treatment of anxiety disorders, a drug for treatment of psychosis, a drug for treatment of mania, a drug for treatment of epilepsy, a drug for treatment of central nervous system degenerative disorders, an analgesics, an opioid analgesic, a drug for treatment of drug addiction, a drug for treatment of drug abuse, and combinations thereof. The active substance that can be delivered by the cryo-cracking treatments described herein can be a muscle relaxant. The muscle relaxant can be selected from the group consisting of a peripherally acting muscle relaxant, a centrally acting muscle relaxant, a directly acting muscle relaxant, a muscle relaxant acting on smooth muscle, a muscle relaxant acting on skeletal muscle, an unclassified muscle relaxant, and combinations thereof. The muscle relaxant can be selected from the group consisting of alcuronim, amyl nitrate, atacurium, baclofen, benzodiazepine, botulinum toxin, carisoprodol, chlormezanone, chlorzoxazone, cisatrcurium, curare, cyclobenzaprine, dantrolene, decamethonium, dimethyltubocurarine, doxacurium, doxacurium chloride, emylcamate, fazadinium, fazadinium bromide, febarbamate, flavoxate, fludiazepam, flunitazepam, flurazepam, gallamine, gidazepam, halazepam, hexafluoronim, loprazolam, lorazepam, lormetazepam, medazepam, mephenesin, meprobamate, metaxalone, methocarbamol, midazolam, mivacurium, mivacurium chloride, nimetazepam, nitrazepam, orphenadine, oxazepam, pancuronium, phenprobamate, phenyramidol, pinazepam, pipercuronium, pipercuronium bromide, prazepam, pridinol, quazepam, rapacuronium, rocuronium, rocuronium bromide, styramate, suxamethonium, suxamethonium chloride, tizanide, temazepam, tetrazepam, thiocolchicoside, tizanidine, tubocurarine, tolperisone, vercuronium, and combinations thereof. The active substance can be a neuromuscular-blocking drug. The neuromuscular blocking drug can be selected from the group consisting of an inhibitor of acetylcholinesterase, succinylcholine, suxamethonium, decamethonium, curare, turbocurarine, atracurium, cisatacurium, vecuronium, rocuronium, mivacurium, pancuronium bromide, a form of boxulinum toxin, and combinations thereof.

The active substance that can be selected from the group consisting of a drug affecting renal function, a drug affecting cardiovascular function, and combinations thereof. The active substance can be selected from the group consisting of a diuretic, vasopressin, an agent affecting the renal conservation of water, renin, angiotensin, a drug for treatment of myocardial ischemia, an antihypertensive agent, a drug for treatment of hypertension, a drug for treatment of heart failure, an antiarrhythmic drug, a drug for treatment of hypercholesterolemia, a drug for treatment of dyslipidemia, and combinations thereof. The active substance that can be delivered by the cryo-cracking treatments described herein can be a drug affecting gastrointestinal function. The drug affecting gastrointestinal function can be selected from the group of a drug for control of gastric acidity, a drug for treatment of peptic ulcers, a drug for treatment of gastrointestinal reflux, a prokinetic agent, an antiemetic, a drug for treatment of irritable bowel syndrome, a drug used to treat diarrhea, a drug used to treat constipation, a drug used to treat inflammatory bowel disease, a drug for treatment of biliary disease, a drug for treatment of pancreatic disease, and combinations thereof. The active substance that can be delivered by the cryo-cracking treatments described herein can be a drug for treatment of urogenital disorders or sexual dysfunction.

The active substance that can be a drug for treatment of parasitic infections. The drug for treatment of parasitic infections can be selected from the group consisting of a drug for treatment of protozoal infections, a drug for treatment of malaria, a drug for treatment of amebiasis, a drug for treatment of giardiasis, a drug for treatment of trichomoniasis, a drug for treatment of trypanosomiasis, a drug for treatment of leishmaniasis, a drug for treatment of helminthiasis, and combinations thereof. The active substance that can be delivered by the cryo-cracking treatments described herein can be a drug for treatment of microbial diseases. The drug for treatment of microbial diseases can be selected from the group consisting of a sulfonamide, trimethoprim-sulfamethoxazole, a quinolone, a drug for treatment of urinary tract infections, a penicillin, a cephalosporin, a .beta.-lactam antibiotic, an aminoglycoside, a protein synthesis inhibitor, an antibacterial agent, a drug for treatment of tuberculosis, a drug for treatment of Mycobacterium avium complex disease, a drug for treatment of leprosy, an antifungal agent, an antiviral agent, an antiretroviral agent, and combinations thereof. The drug for treatment of bacterial infections can be selected from the group consisting of tetracycline, doxycycline, minocycline, erythromycin, trimethoprim, sulfamethoxazole, clindamycin, mupirocin, silver sulfadiazine, and combinations thereof.

The following examples are intended to illustrate but not limit the disclosure. While they are typical of those that might be used, other procedures known to those skilled in the art may alternatively be used.

### EXAMPLES

A study comprising applying conjugated FITC-Dextran with 4000 Daltons molecular weight as a surrogate of water-soluble drugs on ex vivo pork skin samples was performed.

A range of temperature ranges can be used, from less than freezing (20° C.) to very low temperatures (−200° C.). A wide range of cooling compounds can be used, including gaseous, liquid or solid compounds, including refrigerants and cryogens. For example R134a (1,1,1,2 tetrafluor-oethane), dichlorodifluoromethane (CCl2 F2), chlorodifluoromethane (CHClF₂), liquid nitrogen, cold water, ice, cold air and any compound capable of cooling skin to near or below freezing temperatures.

Thermoelectric coolers and other cooling devices can be used as well. In these experiments R134a was used as the cryogenic agent. Exposure time can range from minutes to millisecond exposures, depending on the type of coolant used. For example, R-134 has a temperature of −26° C. and was used in 50-500 millisecond busts to cool skin to −10-−15° C.

For applying suction, any method of creating suction or positive pressure on the skin can be used. For example, the Isolaze device from Aesthera can be used to apply a suction of 380 mm Hg.

Suction and cooling can each be applied in combination with phototherapy in various combinations. For example, cooling can precede suction, followed by or simultaneous with phototherapy.

Phototherapy can include broadband light or intense pulsed light (IPL), as well as laser light and even other heating sources along with cooling and suction to enhance perfusion of drugs. Light acts to heat up tissue, dilate pores and thus accelerate the uptake of agents. Any other heat source for this purpose may work.

To investigate this, a study was initiated consisting of applying conjugated FITC-Dextran with 4000 Daltons molecular weight as a surrogate of water-soluble drugs on ex vivo pork skin samples. After exposing skin samples for 30 min to 3 hours after topical application, a cross-sections of skin was cut (˜20 microns thick) to observe directly the Dextran penetration via the intensity of the FITC fluorescence with the aid of a confocal fluorescent microscope.

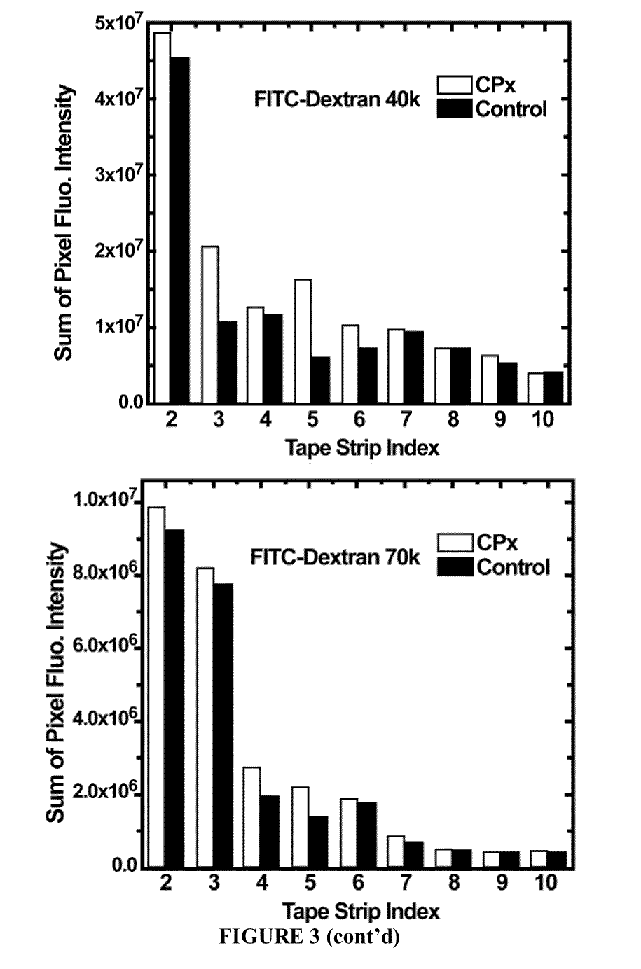

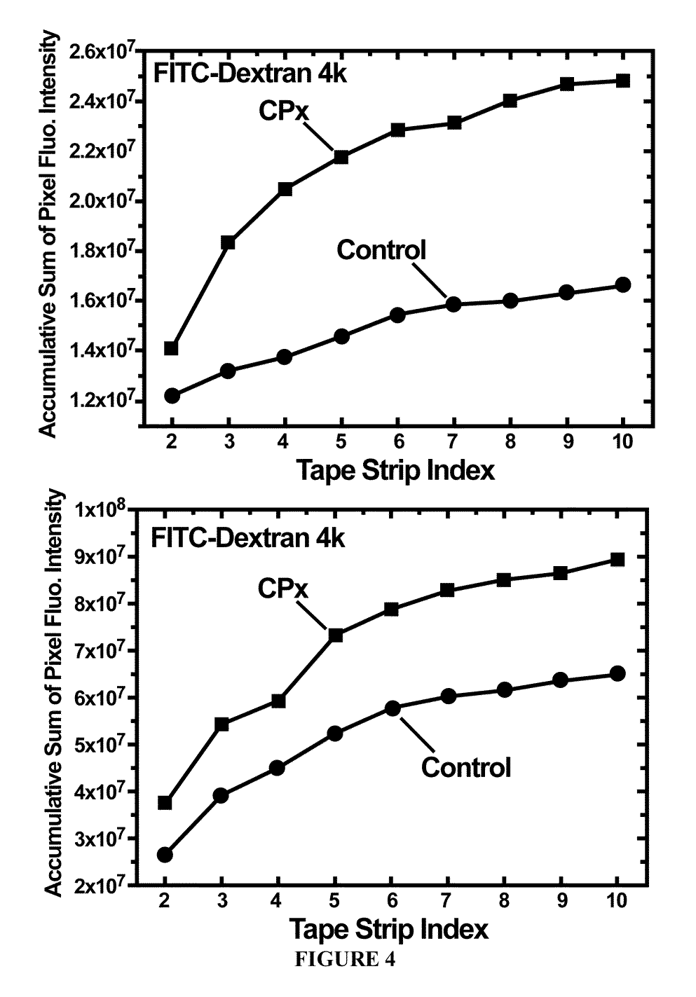

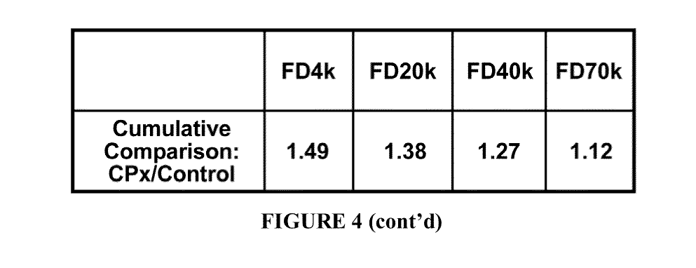

Experimental drug surrogates with various weight (from 500 Daltons to 70,000 Daltons) and hydrophilicity have been tested. For this purpose FITC (500 Daltons), and several combinations of FITC adhered to Dextran of various molecular weights from 4 kDa to 70 kDa but also, Dylight and Curcumin, which have different hydrophilicity were used (see, e.g., FIGS. 3 and 4).

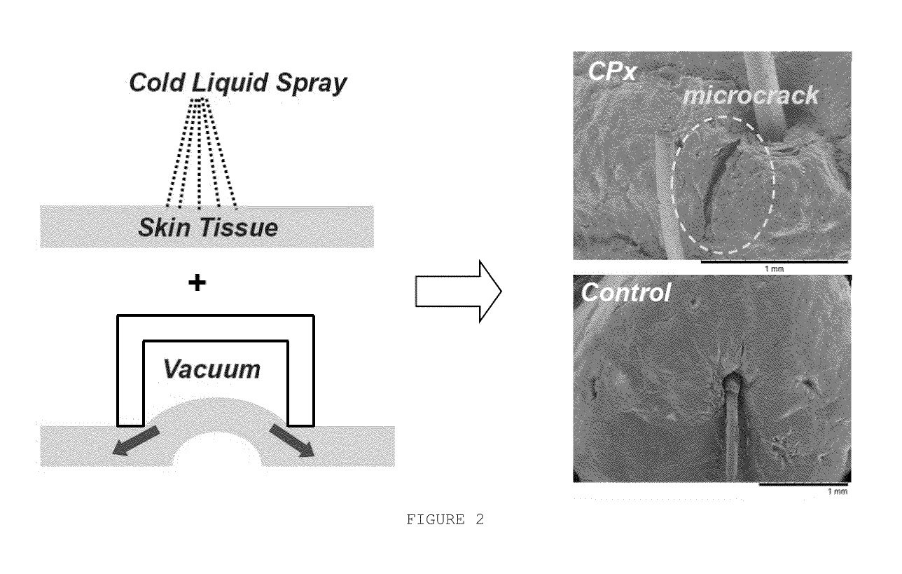

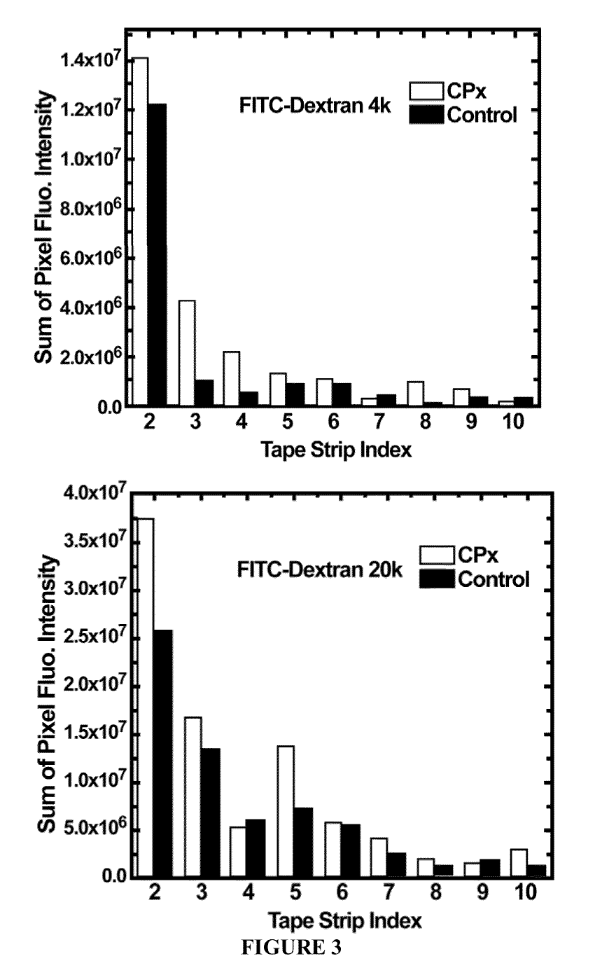

In terms of cryo-cracking technology, electron microscope images to corroborate the “cracking” of the stratum corneum produced by the combined use of cold fluid on the skin followed by the stretching (see, e.g, FIG. 2).

A case clinical study since then, which seems to prove that even as close as 3 hrs after topical application using the procedure described herein, the penetration of FITC into the stratum corneoum and epidermis down the microcracks created by cryocracking is much more significant compared to the penetration after simple rubbing at the same time (see FIG. 5). This study also included FITC with Dextran of 4 kDa and 70 kDA.

It is worth stressing the fact that significant penetration with this drug surrogate was observed, as this is a hydrophilic compound which should have much greater difficulty getting diffused into the skin given the hydrophobicity of skin. Most lotions and creams used for dermatologic purposes use lipophillic vehicles to facilitate diffusion. Thus, showing diffusion improvement with cryocracking relative to rubbing only or even after stretching by the suction cup without cooling is noteworthy.

A number of embodiments of the invention have been described. Nevertheless, it will be understood that various modifications may be made without departing from the spirit and scope of the invention. Accordingly, other embodiments are within the scope of the following claims.

What is claimed is:

1. A method of trans-dermal delivery of a biomolecule comprising:
freezing a location on the skin of a subject;
contacting the location with a pressure to cause deformation of the skin; and
contacting the location with a factor to be delivered.

2. The method of claim 1, further comprising contacting the location with heat prior to, simultaneously with, or immediately after contacting the location with the factor.

3. The method of claim 1, wherein the pressure is negative pressure.

4. The method of claim 3, wherein the negative pressure is a vacuum.

5. The method of claim 1, wherein the method produces micro-cracks or channels in the stratum corneum (SC).

6. The method of claim 5, wherein the micro-cracks/channels extend to a depth below the stratum corneum.

7. The method of claim 6, wherein the factor is delivered into one or more of the microchannels/cracks.

8. The method of claim 7, further comprising sealing the location.

9. The method of claim 5, wherein the factor is a drug.

10. The method of claim 1, wherein the pressure is alternating positive and negative pressure.

11. The method of claim 1, wherein the agent is an optical clearing agent.

12. The method of claim 1, used in combination with heating.

13. The method of claim 12, wherein the heating is by a laser, radio wave or convection.

14. A device comprising a cryogenic freezing means, a pressure delivery means and an drug delivery means.
